(19) United States
(12) Patent Application Publication
(10) Pub. No.: US 2024/0245510 A1
(43) Pub. Date: Jul. 25, 2024

(54) Balloon Nosecone for Delivery Device

(71) Applicant: Cephea Valve Technologies, Inc., Abbott Park, IL (US)

(72) Inventors: Randolf Von Oepen, Aptos, CA (US); Jarred Hake, Fremont, CA (US)

(73) Assignee: Cephea Valve Technologies, Inc., Abbott Park, IL (US)

(21) Appl. No.: 18/397,642

(22) Filed: Dec. 27, 2023

Related U.S. Application Data
(60) Provisional application No. 63/481,017, filed on Jan. 23, 2023.

(51) Int. Cl. A61F 2/24; A61M 25/10

(52) U.S. Cl. CPC A61F 2/2433; A61F 2/2436; A61M 25/1025

(57) ABSTRACT

A delivery system for delivering a medical device includes a delivery device. The delivery device may include an outer delivery sheath having a compartment at a distal end thereof, the compartment configured to receive the medical device. The delivery device may also include a nosecone catheter positioned radially within the outer delivery sheath, the nosecone catheter having an inner tube configured to receive a guidewire therethrough, and an outer tube, an inflation lumen defined between the inner tube and the outer tube. The delivery device may further include a nosecone that has a length that may be adjusted by pushing an inflation medium into the balloon nosecone or withdrawing the inflation medium from the balloon nosecone via the inflation lumen, the balloon nosecone being reversibly coupled to the nosecone catheter.

### CROSS-REFERENCE TO RELATED APPLICATIONS

The present application claims priority to U.S. Provisional Ser. No. 63/481,017, filed Jan. 23, 2023, the disclosure of which is hereby incorporated by reference in its entirety as if fully set forth herein.

### BACKGROUND OF THE DISCLOSURE

The present disclosure relates generally to heart valve replacements, and more particularly to collapsible and expandable prosthetic heart valves. Still more particularly, the present disclosure relates to methods and devices used for delivering and deploying collapsible prosthetic heart valves into a patient.

The mitral valve lies between the left atrium and the left ventricle of the heart, while the tricuspid valve lies between the right atrium and the right ventricle of the heart. Various diseases can affect the function of the mitral and tricuspid valves, including degenerative valve disease and valve prolapse. These diseases can cause valve stenosis, in which the valve fails to open fully and thereby obstructs blood flow, and/or valve insufficiency, in which the valve is incompetent and blood flows passively in the wrong direction.

Many patients with heart disease, such as problems with the mitral or tricuspid valve, are intolerant of the trauma associated with open-heart surgery. Age or advanced illness may have impaired the patient's ability to recover from the injury of an open-heart procedure. Additionally, the high costs associated with open-heart surgery and extra-corporeal perfusion can make such procedures prohibitive.

Patients in need of cardiac valve repair or cardiac valve replacement can be served by minimally invasive techniques. In many minimally invasive procedures, small devices are manipulated within the patient's body under visualization from a live imaging source like ultrasound, fluoroscopy, or endoscopy. Minimally invasive cardiac procedures are inherently less traumatic than open procedures and may be performed without extra-corporeal perfusion, which carries a significant risk of procedural complications.

During minimally invasive procedures for cardiac valve replacement, an appropriate valve prosthesis generally must be collapsed into a small delivery catheter for delivery to and deployment within the native valve annulus. However, prosthetic mitral and tricuspid valves are typically relatively large and may require correspondingly large delivery devices. It would thus be desirable for systems to allow for relatively small delivery devices despite having relatively large prosthetic heart valves carried within the delivery devices.

### BRIEF SUMMARY

According to one aspect of the disclosure, a delivery system for delivering a medical device includes a delivery device. The delivery device may include an outer delivery sheath having a compartment at a distal end thereof, the compartment configured to receive the medical device therein. The medical device may be a prosthetic heart valve, and the compartment may be a valve cover configured to receive the prosthetic heart valve while the prosthetic heart valve is in a collapsed condition. The delivery device may also include a nosecone catheter positioned radially within the outer delivery sheath, the nosecone catheter having an inner tube configured to receive a guidewire therethrough, and an outer tube, an inflation lumen defined between the inner tube and the outer tube. The delivery device may further include a nosecone that has a length that may be adjusted by pushing an inflation medium into the balloon nosecone or withdrawing the inflation medium from the balloon nosecone via the inflation lumen, the balloon nosecone being reversibly coupled to the nosecone catheter.

The prosthetic heart valve may be configured to be loaded into the valve cover in the collapsed condition while the nosecone is decoupled from the nosecone catheter, and the nosecone is configured to be coupled to the nosecone catheter after the prosthetic heart valve is loaded into the valve cover. The nosecone may be formed as a balloon, the balloon having a proximal end fixed to a nosecone adapter. The nosecone catheter may include a catheter adapter at a distal end thereof, the catheter adapter configured to releasably couple to the nosecone adapter. The catheter adapter and the nosecone adapter may be configured to releasably couple via a threaded mechanism. The catheter adapter may include a channel extending therethrough, the channel being in fluid communication with the inflation lumen. The nosecone adapter may include a lumen extending therethrough and leading into an interior volume of the nosecone, the channel being in fluid communication with the lumen when the catheter adapter is coupled to the nosecone adapter. When the catheter adapter is coupled to the nosecone adapter, a first gasket may be positioned between the nosecone adapter and the catheter adapter, and a second gasket may be positioned between the nosecone adapter and the catheter adapter, the first and second gaskets positioned on opposite sides of the channel and on opposite sides of the lumen. The delivery device may further include a suture catheter positioned radially within the outer delivery sheath and at least partially surrounding the nosecone catheter, the suture catheter having a tip ring at a distal end thereof. The delivery system may further include a suture ring, the suture ring defining an interior lumen and a plurality of bores, one or more suture threads extending through the plurality of bores. When the prosthetic heart valve is received within the valve cover in the collapsed condition, the one or more suture threads may be reversibly coupled to the prosthetic heart valve. The suture ring may be configured to reversibly couple to the tip ring. When the suture ring is coupled to the tip ring, both the inner tube and the outer tube of the nosecone catheter may pass through the interior lumen of the suture ring. The suture ring may include a cylindrical body portion and a distal head, the distal head having a generally frustoconical shape that increases in diameter in a distal direction, the plurality of bores being formed in the distal head. The suture ring may include a cylindrical body portion and a distal head, the distal head having a generally hemispherical shape, recesses being formed at locations in the cylindrical body at spaced distances in a circumferential direction of the cylindrical body portion, each of the plurality of bores being formed in the distal head adjacent a corresponding one of the recesses.

According to another aspect of the disclosure, a method comprises introducing a delivery catheter into a femoral vein of a patient while a prosthetic heart valve is contained in a collapsed condition within a valve cover of the delivery catheter. The method may include advancing the delivery catheter through the femoral vein toward a right atrium of the patient while a nosecone at a leading end of the delivery catheter has a first length. Advancement of the delivery catheter may be continued through the femoral vein until the nosecone approaches the right atrium. After the delivery catheter approaches the right atrium, a volume of inflation medium may be withdrawn from the nosecone so that the nosecone has a second length smaller than the first length. While the nosecone has the second length, the valve cover may be maneuvered to a position within or adjacent to an atrioventricular valve of the patient. After maneuvering the valve cover to the position within or adjacent to the atrioventricular valve of the patient, the prosthetic heart valve may be deployed into the atrioventricular valve. The prosthetic heart valve may be a prosthetic mitral valve, and the method may further include advancing the delivery catheter through the right atrium and across an atrial septum while the nosecone has the first length, wherein withdrawing the volume of inflation medium from the nosecone is not performed until the delivery catheter crosses the atrial septum. The prosthetic heart valve may be a prosthetic tricuspid valve, and the volume of inflation medium may be withdrawn from the nosecone upon or immediately after the delivery catheter enters the right atrium. The method may further include, prior to introducing the delivery catheter into the femoral vein, loading the prosthetic heart valve into the valve cover while the nosecone is decoupled from the delivery catheter, and after the prosthetic heart valve is loaded into the valve cover, coupling the nosecone to the delivery catheter. Introducing the delivery catheter into the femoral vein may be performed without passing the delivery catheter through an introducer catheter that has previously been introduced into the femoral vein.

### BRIEF DESCRIPTION OF THE DRAWINGS

FIG. 1 is a perspective view of a frame structure for a prosthetic heart valve.

FIG. 2 is a longitudinal cross-sectional view of a distal end portion of one embodiment of a delivery catheter for a prosthetic heart valve.

FIGS. 3A-B are perspective views of one embodiment of a coupling member of a suture rigging assembly of the present disclosure.

FIG. 4 is a side elevational view of the coupling member of FIGS. 3A-B.

FIG. 5 is a longitudinal cross-sectional view of the coupling member of FIGS. 3A-B.

FIG. 6 is a rear perspective view of the coupling member of FIGS. 3A-B.

FIGS. 7A-B are distal end views of the coupling member of FIGS. 3A-B.

FIGS. 8A-B are proximal end views of the coupling member of FIGS. 3A-B.

FIG. 9 illustrates a tether member.

FIG. 10 is a partial perspective view of the suture rigging assembly according to the present disclosure, including the coupling member of FIGS. 3A-B and a plurality of the tether members of FIG. 9 attached to the coupling member.

FIG. 11 is an enlarged partial view of the suture rigging assembly showing the attachment of the tether members to the coupling member.

FIG. 12 is a proximal end view showing the attachment of a padding suture beneath the suture threads of the tether members attached to the coupling member.

FIG. 13 is a perspective view of the suture rigging assembly showing radiopaque markers attached to the tether members.

FIG. 14 is a perspective view of the suture rigging assembly attached between the delivery catheter and a prosthetic heart valve.

FIGS. 15A-B are schematic views of a distal end of a delivery device having relatively short and long nosecones, respectively.

FIG. 15C is a computed tomography (“CT”) scan image of the right heart.

FIG. 15D is a CT scan image of the right heart with a representative valve cover extending through the valve annulus into the ventricle.

FIG. 16A is a longitudinal cross-section of a distal end of a delivery device that includes a balloon nosecone coupled to the delivery device by a specialized adapter.

FIGS. 16B and 16C are longitudinal cross-sections, respectively, of the distal end of the nosecone catheter and the proximal end of the balloon nosecone of FIG. 16A.

FIGS. 17A-F are perspective views of alternate designs of the coupling member of FIGS. 3A-8B.

### DETAILED DESCRIPTION

As used herein, the terms “proximal” and “distal,” when used in connection with a delivery device or components of a delivery device, including a suture rigging assembly, are to be taken as relative to a user of such device. “Proximal” is to be understood as relatively close to the user when the device is being used as intended, and “distal” is to be understood as relatively far away from the user when the device is being used as intended. As used herein, the terms “substantially,” “generally,” “approximately,” and “about” are intended to mean that slight deviations from absolute are included within the scope of the term so modified.

The suture rigging assembly described herein can be used to attach a wide variety of prosthetic heart valves to a catheter-based delivery device, to load the prosthetic heart valve into the delivery device, and/or to sustain a tensile load path between the prosthetic heart valve and the delivery device until the valve is deployed in a patient. Exemplary prosthetic heart valves that can be used with the suture rigging assembly described herein include the expandable prosthetic heart valves described in U.S. Patent Publication No. 2016/0158000; in U.S. Pat. No. 8,870,948; and in PCT Publication No. WO 2016/183526, the disclosures of all of which are hereby incorporated by reference herein. For example, the suture rigging devices described herein are configured for use with prosthetic heart valves, such as prosthetic mitral or tricuspid valves, having pins to which tethers of the suture rigging assembly attach.

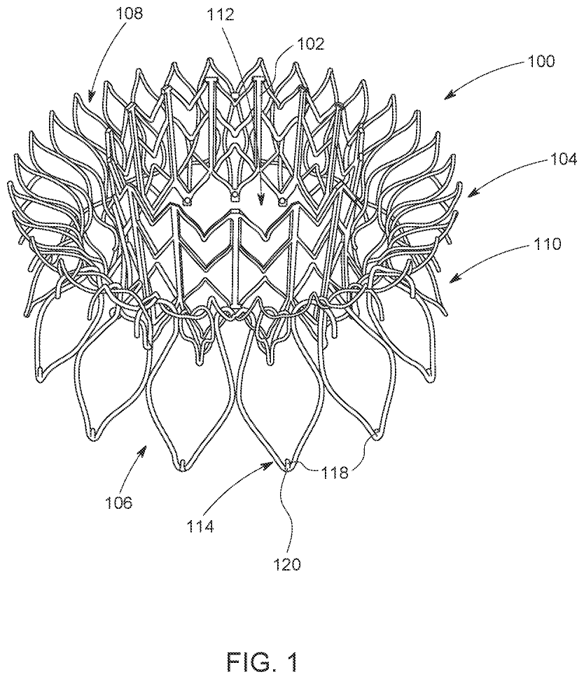

FIG. 1 is a perspective view of the frame structure for an exemplary prosthetic heart valve 100. Prosthetic heart valve 100 may be a prosthetic mitral valve having an expandable and collapsible frame structure that includes an inner strut frame 102 surrounded by an outer anchor assembly 104. However, prosthetic heart valve 100 may be suitable for replacing other native heart valves, such as the tricuspid valve, the aortic valve or the pulmonary valve. Anchor assembly 104, shown in an expanded state, includes an atrial anchor 106 configured to be positioned on the atrial side of the native mitral valve annulus, a ventricular anchor 108 configured to be positioned on the ventricular side of the native mitral valve annulus, and a central portion 110 positioned axially between the atrial anchor and the ventricular anchor. Anchor assembly 104 may have an hourglass shape in the expanded state in that each of the atrial anchor 106 and the ventricular anchor 108 flares radially outward of the central portion 110, such that the central portion defines a waist between the atrial anchor and the ventricular anchor. Strut frame 102 may be positioned radially inward of anchor assembly 104 and may be formed of a plurality of interconnected struts. The radially inner surface of strut frame 102 defines a perimeter of a central opening 112, which enables blood to flow through prosthetic heart valve 100.

Prosthetic heart valve 100 includes one or more leaflets (not shown) that may be secured to strut frame 102 and disposed at least partially in central opening 112. The leaflets are configured to coapt with one another to control blood flow through the prosthetic heart valve, allowing blood to flow from the atrial anchor 106 toward the ventricular anchor 108 (the antegrade direction), but substantially blocking blood from flowing in the opposite (retrograde) direction. In some embodiments, one or more skirts or cuffs (not shown) may partially or fully cover inner and/or outer surfaces of anchor assembly 104 and/or strut frame 102. Such skirts or cuffs may be formed from fabric and/or tissue materials, for example.

Both the atrial anchor 106 and the ventricular anchor 108 of anchor assembly 104 may include a plurality of petals or cells 114 that are joined to one another around the circumference of the anchor assembly. When prosthetic heart valve 100 is in a fully expanded state, the petals or cells 114 on both atrial anchor 106 and ventricular anchor 108 are fully extended radially outward, as shown in FIG. 1. Prosthetic heart valve 100 is naturally in an expanded state when no force is applied to the petals or cells 114. The petals or cells 114 of anchor assembly 104 may be configured to collapse and/or to reduce the outer diameter of the frame structure when the frame structure is loaded into a delivery device. When prosthetic heart valve 100 is in a collapsed state, the petals or cells 114 on both atrial anchor 106 and ventricular anchor 108 are at least partially collapsed radially inward. Prosthetic heart valve 100 may be placed in the collapsed state by applying pressure to petals or cells 114 in a radially inward direction.

The petals or cells 114 on atrial anchor 106 (and/or ventricular anchor 108) may include a pin 118 or other attachment member to which tether loops may be connected, as will be described below. Pins 118 may be attached to or formed on some or all of the petals or cells 114 on atrial anchor 106 (and/or ventricular anchor 108) and are sized and shaped so that the tether loops remain attached when under tension but are configured to be released after the deployment of prosthetic valve 100 within the patient. As shown in FIG. 1, pins 118 may be provided at the apex 120 of each petal or cell 114 on atrial anchor 106. However, this need not be the case and pins 118 may be provided on less than all of the petals or cells 114 of atrial anchor 106, on some or all of the petals or cells of ventricular anchor 108, or at other locations on anchor assembly 104.

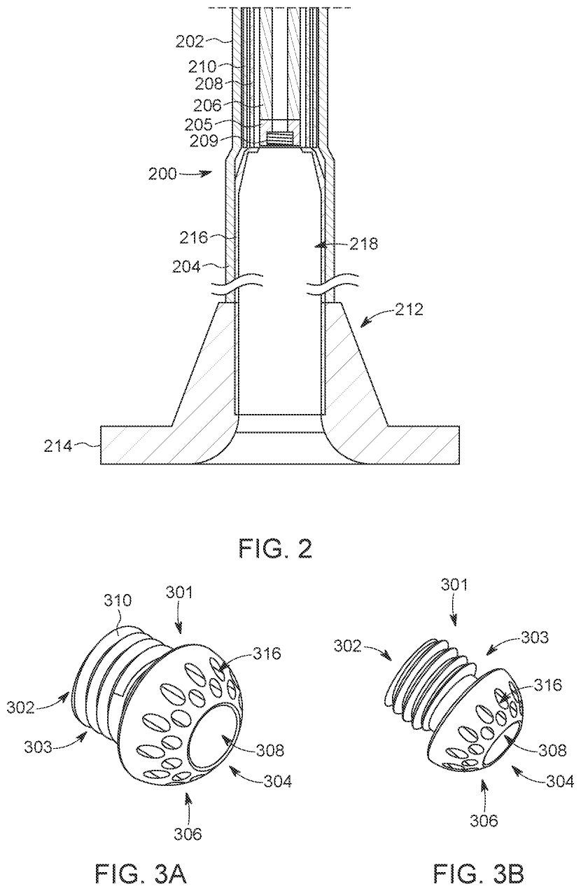

A portion of the distal end of a delivery device 200 for delivering and deploying prosthetic heart valve 100 within a patient is shown in FIG. 2. Delivery device 200 may include an outer delivery sheath 202. At its distal end, outer delivery sheath 202 may have a portion that serves as a valve cover 204 for receiving prosthetic heart valve 100 and maintaining it in a collapsed condition until it is deployed. In one example, the portion of the outer delivery sheath 202 that serves as the valve cover 204 can have an enlarged diameter compared to the proximal end, as depicted in FIG. 2, while in other examples, the distal and proximal portions of the outer delivery sheath 202 can have the same or substantially the same diameter. The valve cover 204 may be generally referred to as a sheath or a compartment for receiving a medical device. In one embodiment, a suture catheter 206, an extension catheter 208, and a steering catheter 210 may be located coaxially within outer sheath 202 and may be slidable relative to one another. Delivery device 200 may also include a guidewire (not shown) positioned within and extending through the lumen of a nosecone catheter received within the suture catheter 206. The nosecone catheter may have a distal end that is coupled to an atraumatic tip or nosecone after the prosthetic heart valve 100 is loaded into the valve cover 204. A control handle (not shown) may be attached to the proximal ends of suture catheter 206, extension catheter 208, steering catheter 210 and outer delivery sheath 202, and may be used to manipulate and control movement of these various components relative to one another to perform various functions. Suture catheter 206 may include a tip ring 205 at its distal end. Tip ring 205 may include internal threads 209 for threaded engagement with suture rigging assembly 300, as described below. Other mechanisms for attaching suture rigging assembly 300 to delivery device 200 are also contemplated herein.

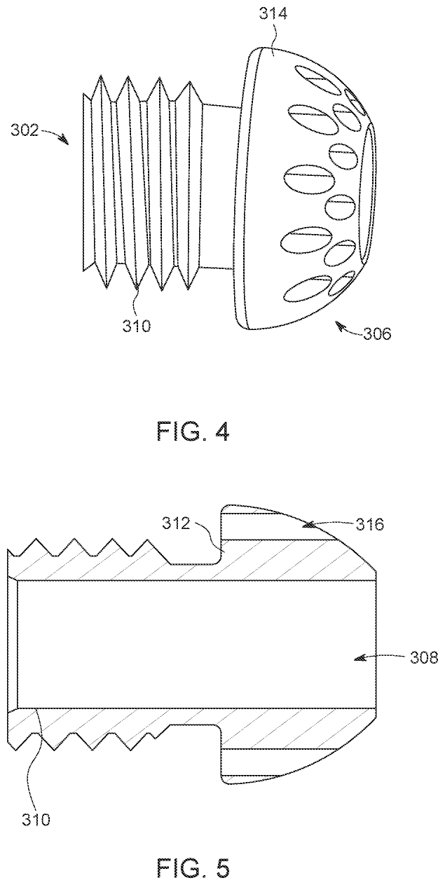

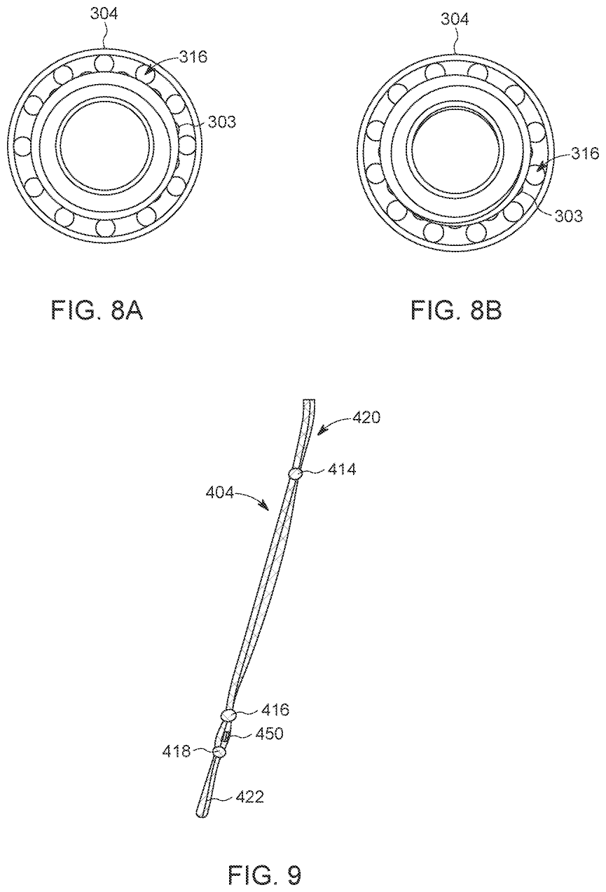

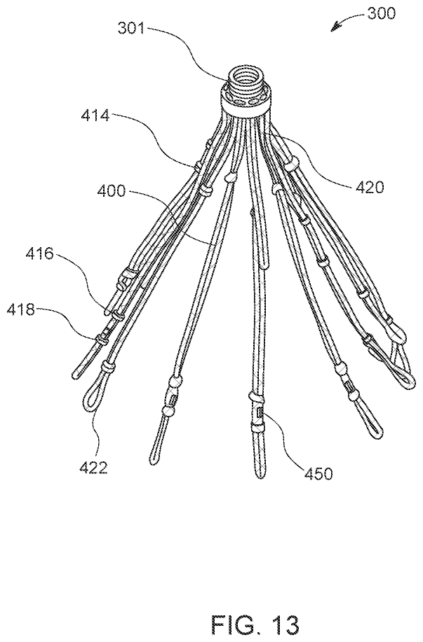

A suture rigging assembly 300 that assists in collapsing and drawing prosthetic heart valve 100 into delivery device 200 is shown in FIG. 13. Suture rigging assembly 300 includes a coupling ring 301 to which a plurality of suture tethers may be attached. One embodiment of coupling ring 301 is illustrated in FIGS. 3-8. As shown, coupling ring 301 extends in a longitudinal direction between a distal end 306 and a proximal end 302, and has somewhat of a mushroom shape with a generally cylindrical body 303 at the proximal end terminating in an enlarged head 304 at the distal end. A lumen 308 may extend in the longitudinal direction through the cylindrical body 303 and head 304 of coupling ring 301 and may be sized to receive a component of a delivery device, such as the nosecone catheter (and any guidewire received through the nosecone catheter) or another structure therethrough.

As illustrated, cylindrical body 303 may be formed with external threads 310 that are sized and shaped to securely connect to the internal threads 209 in the tip ring 205 of suture catheter 206. However, as mentioned, the present disclosure contemplates other fasteners for attaching coupling ring 301 to delivery device 200. In one example, the cylindrical body 303 of coupling ring 301 could be formed with internal threads designed to mate with external threads on suture catheter 206 or another component of delivery device 200. In another example, the cylindrical body 303 of coupling ring 301 may be eliminated, and the internal threads could be formed within the enlarged head 304 of the coupling ring. In still another embodiment, coupling ring 301 could include a transverse pin or a pair of aligned bosses protruding from opposite sides of cylindrical body 303 that are configured to mate with corresponding undercut recesses formed in suture catheter 206 or another component of delivery device 200. Other fastening mechanisms, including, but not limited to, a snap connection mechanism, are also contemplated so long as they are sufficiently strong to withstand the substantial tensile forces that will be exerted thereon as prosthetic heart valve 100 is collapsed and loaded into delivery device 200.

The head 304 of coupling ring 301 has a diameter that is substantially larger than the diameter of cylindrical body 303, thereby defining a shoulder 312 extending around the cylindrical body and facing toward the proximal end 302 of the coupling ring. Head 304 may have a domed or hemispherical surface 314 facing away from the proximal end 302 of coupling ring 301, the purpose of which will be explained below. Other smoothly curved surfaces are also possible, including elliptical, oval, oblong and the like. A plurality of round apertures or bores 316 may extend through head 304 from shoulder 312 to surface 314. Bores 316 may extend parallel to one another and parallel to the longitudinal direction of coupling ring 301, and each has a diameter sized to receive a length of suture thread. Bores 316 may extend in two rings in an annular direction around the central longitudinal axis of coupling ring 301, an inner ring 318 and an outer ring 320. In the illustrated embodiment, coupling ring 301 has twenty-four bores, with twelve bores in inner ring 318 and twelve bores in outer ring 320. However, coupling ring 301 may have more or less than twenty-four bores, and the number of bores in the inner and outer rings need not be the same. Further, bores 316 need not be arranged in concentric rings, but may be arranged in any pattern that will avoid the suture threads becoming entangled with one another when assembled to coupling ring 301.

As a result of the curvature of surface 314, the bores 316 in inner ring 318 will define an elliptical shape with a relatively small major axis where they intersect surface 314. The bores 316 in outer ring 320, on the other hand, will define an elliptical shape with a larger major axis where they intersect surface 314. The major axes of the ellipses defined by the bores 316 in both inner ring 318 and outer ring 320 extend in directions radially outward from the central longitudinal axis of coupling ring 301. This arrangement enables the suture threads 400 that extend through bores 316 to fan radially outward and may minimize contact with a sharp edge or corner of coupling ring 301.

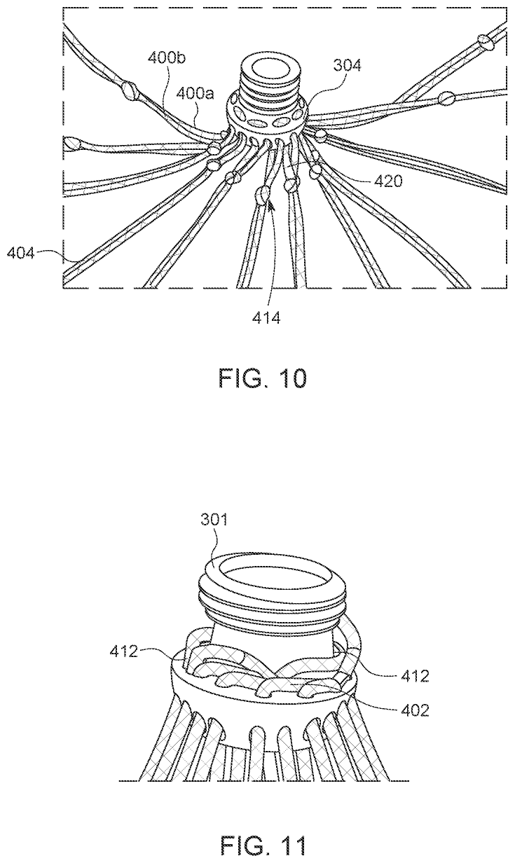

One or more suture threads 400 may be attached to the head 304 of coupling ring 301. The suture threads 400 may be comprised of various materials, both man-made and natural. Examples of natural suture materials may include, but are not limited to, silk, linen, and catgut. Examples of synthetic suture materials may include, but are not limited to, textiles such as nylon or polyester, or flexible metallic cables. Referring to FIGS. 9-13, an elongated suture thread 400 may be threaded through a plurality of the bores 316 in coupling ring 301 to form tethers 404. Suture rigging assembly 300 may include a coupling ring 301 having at least one tether 404 or a plurality of tethers. For example, suture thread 400 may be threaded distally through a bore 316 in inner ring 318 and then proximally though an adjacent bore in outer ring 320, thereby forming an elongated loop or tether 404 extending distally from coupling ring 301. Thus, tether 404 includes two lengths of suture thread 400a and 400b extending side-by-side and continuous with one another at their distal ends. Suture thread 400 may then be threaded distally through an adjacent bore 316 in inner ring 318 and then proximally through an adjacent bore in outer ring 320, thereby forming another elongated loop or tether 404 extending distally from coupling ring 301. This pattern may be repeated to form a plurality of elongated loops or tethers 404 around the entire circumference of coupling ring 301. Thus, tethers 404 may be formed by a single continuous suture thread 400, with the leading and trailing ends of the suture thread being joined to one another by one or more terminating knots 412. Multiple terminating knots 412 may be used to create a more compact configuration. Specifically, leading and trailing lengths of suture thread 400 may be secured together with a first terminating knot 412 spaced from the ends of the suture thread, resulting in two remaining lengths of the suture thread leading from the first terminating knot 412. These remaining lengths may then be wrapped around the cylindrical body 303 of coupling ring 301, pulling the first terminating knot closer to coupling ring 301. The ends of suture thread 400 may then be secured with a second terminating knot 412. Any remaining lengths of the suture thread may then be trimmed away from the second terminating knot 412. Using multiple knots in this way may decrease the protrusions of suture thread 400 from the cylindrical body 303 of coupling ring 301 and may additionally result in a more secure attachment between the suture thread and the coupling ring. Alternatively, tethers 404 may be formed by multiple suture threads 400, with each suture thread forming plural tethers, and with the adjacent ends of the threads being joined to one another by a terminating knot 412. In a still further alternative, a large knot may be formed at the ends of the suture thread that cannot be pulled through bores 316.

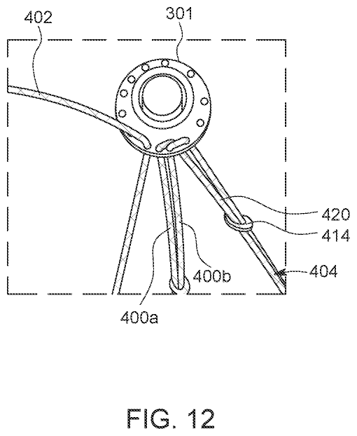

As suture threads 400 are being attached to coupling ring 301, a length 402 of suture thread optionally may be positioned around the circumference of the coupling ring and captured between the shoulder 312 of head 304 and the loops of the suture thread being threaded through bores 316, all as shown in FIGS. 11 and 12. Padding suture thread 402 may simply be an additional length of suture thread 400. In such an event, padding suture thread 402 may be a leading or trailing end of the suture thread threaded through bores 316 to form tethers 404. Padding suture thread 402 may simply be held in place between the shoulder 312 of head 304 and the suture thread loops, or may be tied off with the opposite end of suture thread 400 with terminating knots 412. If padding suture thread 402 is formed from a material with a different diameter than suture thread 400, or from a separate length of suture thread 400, the padding suture thread may be knotted to itself with a knot that may sit under the terminating knot 412 securing the tethers. In instances where the diameter of padding suture thread 402 is the same as the diameter of tethers 404, the padding suture thread may be secured with a terminating knot 412. Padding suture thread 402 may protect suture threads 400 from damage due to contact with any sharp edges on coupling ring 301 and may otherwise reduce stress concentrations in tethers 404 at their interface with the coupling ring when under load, thereby enabling each of the tethers to exhibit an increased tensile capacity. The inclusion of padding suture thread 402 can result in between a 10 and 20 percent increase in the tensile capacity of suture rigging assembly 300.

Suture threads 400a and 400b forming a tether 404 may be joined together by a first knot or stop knot 414 at a spaced distance from coupling ring 301. Stop knots 414 reduce the ability of suture threads 400a and 400b to separate too far from one another or to create a large loop or lasso. The distance between the knots on a tether 404 will define the maximum loop or lasso that can be formed by the tether. As a result of using knots, any loop or lasso able to form will be smaller in size than the loop or lasso in a tether 404 that does not have any knots. Preventing the formation of large loops or lassos is important because a large loop or lasso may become entangled with the apexes of the ventricular anchor 108, thereby impairing the user's ability to pull back the entangled tether 404 after valve 100 has been deployed. As shown in FIG. 13, stop knots 414 in adjacent tethers 404 preferably are formed at different spaced distances from coupling ring 301. For example, the stop knots 414 in a first group of tethers 404 may be spaced a first distance from coupling ring 301, and the stop knots in a second group of tethers that alternate with the tethers in the first group may be spaced a second distance greater than the first distance from the coupling ring. By offsetting the stop knots 414 in adjacent tethers 404 from one another, the tethers are better able to collapse to a smaller, more compact cross-sectional size within the confines of delivery device 200.

The stop knots 414 in tethers 404 create in each tether an upper or proximal connecting loop 420 between the knot and coupling ring 301. The ability of suture thread 400 to move freely within bores 316 enables the lengths of tethers 404 to self-adjust to a certain degree. That is, each tether 404 is free to move proximally until its stop knot 414 contacts coupling ring 301 and is free to move distally until the stop knots in the adjacent tethers contact the coupling ring. Therefore, as one tether 404 lengthens as it moves distally, there is a corresponding proximal movement and shortening of the adjacent tethers on either side of it, in the manner of a pulley. This adjustment in the lengths of tethers 404 enables a balancing of the load imparted to each of the tethers as prosthetic heart valve 100 is collapsed during loading into delivery device 200 or during re-sheathing. For example, if a shorter tether 404 experiences a higher tensile stress upon the loading of prosthetic heart valve 100 into delivery device 200, that tether may lengthen as the adjacent tethers shorten until the tensile stress on all of the tethers reaches an equilibrium point at which the total tensile stress is substantially evenly distributed among all of the tethers. Maintaining a balanced load among tethers 404 prevents any one of the tethers from becoming overloaded and breaking, which can impede the functionality of the entire system. Further, more evenly distributing the load among tethers 404 enables the overall tensile capacity of suture rigging assembly 300 to be increased.

Additional knots may also be formed at the distal or closed end of tethers 404. As shown in FIG. 13, a third knot or lower fixture knot 418 may be formed at a spaced distance from the distal end of tether 404, forming a closed attachment loop 422 therein. Attachment loops 422 are intended to hook onto the pins 118 of prosthetic heart valve 100 and to apply tension to assist in collapsing the prosthetic heart valve during loading into delivery device 200, as described more fully below. Preferably, attachment loops 422 have a relatively small size so that, following their release from pins 118 during deployment, they do not become entangled with the pins or other structures of prosthetic heart valve 100, impeding proper deployment of the heart valve and removal of delivery device 200 from the patient.

To help visualize the locations of tethers 404, and in particular the positions of attachment loops 422, during the deployment of prosthetic heart valve 100 in a patient, some embodiments of suture rigging assembly 300 may include a radiopaque marker 450 on all or at least some of the tethers. Radiopaque markers 450 may be formed of any material that can be readily visualized under fluoroscopy, including metals such as gold, platinum, platinum-iridium, tantalum, tantalum-tungsten, and others, and may take any shape. Preferably, radiopaque markers 450 have a bore or channel extending therethrough so that the markers may be threaded onto suture threads 400a and/or 400b before lower fixture knot 418 is formed therein or as suture thread 400 is threaded through bores 316. In some embodiments, radiopaque markers 450 may be cylindrical, with a bore extending therethrough along the longitudinal axis of the cylinder. The radiopaque markers 450 provided on suture rigging assembly 300 need not all have the same shape, and different shapes may be assembled to various tethers 404 to indicate the orientation of prosthetic heart valve 100 or to identify various portions thereof. Moreover, if any of tethers 404 is improperly affixed to prosthetic heart valve 100 or becomes improperly affixed to the prosthetic heart valve during delivery of the heart valve into the patient or during deployment, radiopaque markers 450 may help to identify which of the tethers is improperly affixed and identify its location.

Radiopaque markers 450 may be held in a fixed position on tethers 404 by lower fixture knot 418 at the distal end of the marker and by a second or upper fixture knot 416 formed in the tether at the proximal end of the marker. Fixture knots 416 and 418 capture the radiopaque marker 450 therebetween and prevent it from sliding along the length of tether 404 toward or away from attachment loop 422. As a less preferable alternative, adhesives can be used to attach the radiopaque markers 450 at fixed positions to tethers 404. As a result, once a radiopaque marker 450 has been identified under fluoroscopy, the user will know the position of the attachment loop 422 associated with that marker.

The use of knots to form suture rigging assembly 300 provides several advantages. Firstly, it enables adhesives to be avoided, reducing sterilization, storage and biocompatibility issues that adhesives may create. The elimination of adhesives may also reduce the formation of very small particles during the use of delivery device 200, which particles could potentially be released into the patient's bloodstream. The use of knots throughout suture rigging assembly 300 also enables the assembly to be self-balancing, minimizing the tensile stress in any one tether 404 and increasing the overall tensile capacity of the suture rigging assembly. Finally, the various knots in each tether 404 keeps suture threads 400a and 400b close to one another to prevent undesirable entanglement of the tethers with structures of prosthetic heart valve 100 during deployment.

Suture rigging assembly 300 can be used to attach, load, and release a wide variety of heart valves to/from a wide variety of catheter-based delivery systems. Thus, suture rigging assembly 300 is designed to attach to a prosthetic heart valve and sustain a tensile load path between the heart valve and a delivery device as the heart valve is retracted into a sheath of the delivery device.

One way in which suture rigging assembly 300 may be used to collapse and load prosthetic heart valve 100 into the valve cover 204 of delivery device 200 will now be described. Initially, suture rigging assembly 300 is attached to prosthetic heart valve 100. This is accomplished by fitting some or, preferably, all of the attachment loops 422 at the distal ends of tethers 404 over respective pins 118 on prosthetic heart valve 100. Although this is described here as an initial step, it need not be the first step in the process. Suture rigging assembly 300 may be attached to delivery device 200 first, as described below, followed by the attachment of prosthetic heart valve 100 to the suture rigging assembly.

Referring back to FIG. 2, a loading funnel 212 that can be used to help load prosthetic heart valve 100 into valve cover 204 is shown attached to the distal end of the valve cover. In one embodiment, loading funnel 212 may include a funnel portion 214 located at its distal end and an elongated tubular portion 216 located at its proximal end. Funnel portion 214 may smoothly transition from a relatively large diameter at its distal end to a relatively small diameter where it meets tubular portion 216. The diameter at the distal end of funnel portion 214 is preferably larger than the outer diameter of prosthetic heart valve 100 in its expanded condition, and the diameter of the funnel portion where it meets tubular portion 216 is preferably about the same as the diameter of the lumen 218 of the tubular portion. The outer diameter of tubular portion 216 is preferably slightly smaller than the inner diameter of valve cover 204 and the length of the tubular portion may be about the same as the length of the valve cover, such that the tubular portion can be selectively inserted into and nest within the valve cover. Funnel portion 214 may include a plurality of slots (not shown) extending longitudinally along its inner surface. These slots are intended to accommodate ventricular anchoring tines of prosthetic heart valve 100 and prevent them from scraping against the funnel as the heart valve is being collapsed.

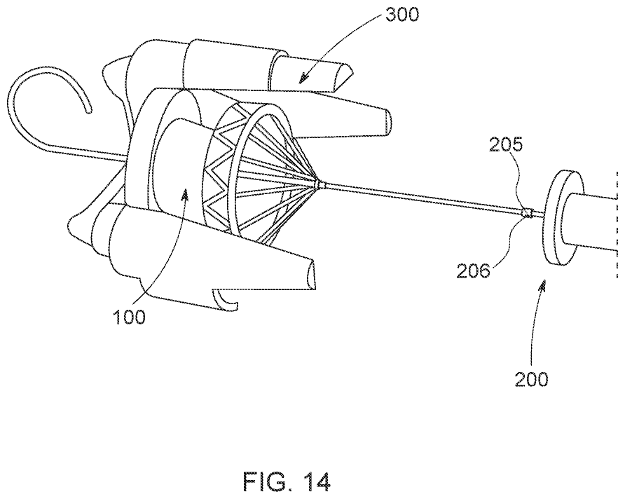

With the tubular portion 216 of loading funnel 212 positioned within valve cover 204, controls located on the operating handle of delivery device 200 may be manipulated to cause suture catheter 206 to advance distally relative to the other components of the delivery device until the tip ring 205 of the suture catheter extends distally beyond the distal end of the tubular portion and into the interior of funnel portion 214. At that point, the threads 310 of coupling ring 301 may be threaded into the threaded portion 209 of tip ring 205 at the distal end of suture catheter 206. FIG. 14 illustrates suture rigging assembly 300 connected to prosthetic heart valve 100, while the prosthetic heart valve 100 is still in a packaging element, and aligned for connection to the suture catheter 206 of delivery device 200. Suture catheter 206 may then be retracted proximally, drawing suture rigging assembly 300 and prosthetic heart valve 100 proximally along with it. As proximal movement continues, tethers 404 are drawn into the lumen of the tubular portion 216 of loading funnel 212. This, in combination with the sloping walls of funnel portion 214, causes the petals or cells 114 on atrial anchor 106 to collapse toward the central axis of prosthetic heart valve 100 and, eventually, to enter the lumen 218 of tubular portion 216. Further proximal movement of suture catheter 206 continues until the petals or cells 114 on ventricular anchor 108 also collapse toward the central axis of prosthetic heart valve 100 and the prosthetic heart valve is completely collapsed and completely positioned within the lumen 218 of the tubular portion 216. At that juncture, while maintaining tension on suture catheter 206, loading funnel 212 may be removed from valve cover 204, leaving the fully collapsed prosthetic heart valve 100 positioned completely within the valve cover. An atraumatic tip (not shown) of the delivery device may then be coupled to the nosecone catheter and retracted to enclose the open distal end of valve cover 204.

In another embodiment, the loading funnel may have a generally cylindrical shape with internal threads at one end and an internal diameter that is about the same as the inner diameter of valve cover 204. The internal threads may mate with external threads at the free end of valve cover 204 to join the loading funnel to the valve cover. A smooth radius on the lumen at the free end of the funnel may help to guide prosthetic heart valve 100 into the funnel lumen.

Once properly loaded, delivery device 200 may be inserted into a patient and directed to a target location, such as the mitral valve annulus, at which prosthetic heart valve 100 may be deployed. To deploy prosthetic heart valve 100, valve cover 204 is retracted proximally over valve 100 while the valve is maintained in position by extension catheter 208. The ventricular anchor 108 of valve 100 will then begin to expand until only the proximal end of the valve (i.e., atrial anchor 106) is held in a collapsed condition by a small cup at the distal end of extension catheter 208. The accurate positioning and orientation of prosthetic heart valve 100 may then be confirmed, after which suture catheter 206 may be advanced distally, relieving tension in tethers 404 and allowing atrial anchor 106 to escape from the cup at the distal end of extension catheter 208 and expand. Suture catheter 206 may be advanced further through the expanded prosthetic heart valve until tethers 404 slip off of pins 118. Suture catheter 206 may then be retracted back into outer delivery sheath 202, the atraumatic tip may be retracted to again close the open end of valve cover 204, and delivery device 200 may be removed from the patient.

As noted above, for transcatheter prosthetic heart valve replacement, it is typically desirable for the delivery device to be as small as possible while still being able to perform the required delivery functions, including housing the collapsed valve during delivery. Prosthetic mitral and tricuspid valves are typically relatively large compared to prosthetic aortic and pulmonary valves, and are typically delivered through the femoral vein. By having a relatively small delivery catheter, it may be possible to avoid a surgical cutdown of the femoral vein. For example, one target for catheter size for a prosthetic mitral or tricuspid valve delivery may be 33 French (11 mm) or lower, preferably 30 French (10 mm) or lower.

Some prosthetic heart valves are delivered with the aid of an introducer. In other words, an introducer tube with a dilator inside the introducer may be passed into the femoral vein prior to introducing the prosthetic heart valve (or the delivery device housing the prosthetic heart valve) into the patient. The dilator of the introducer typically has a long, tapered tip which gently expands the femoral vein as it is pushed through the femoral vein. For example, the tip of the dilator in the introducer can be as long as 10 cm or more, and is typically pushed over a guidewire (e.g. a 0.035 inch wire). After the introducer is in place, the dilator is removed and other devices, such as the delivery device housing the prosthetic heart valve, can be passed through the introducer, with the introducer providing relatively easy access to the femoral vein. Although an introducer may provide certain benefits, it may have disadvantages as well. For example, because the prosthetic heart valve delivery device must pass through the introducer, the combined size of the introducer and delivery device will always be greater than the size of the delivery device alone. In other words, if it is desired to have a catheter size no greater than 30 French (10 mm) at the access site, the introducer may have an outer diameter of 33 French (11 mm) or preferably 30 French (10 mm), but the delivery device may need to have an outer diameter of around 26 French (8.67 mm) in order to be able to fit within the introducer. In other examples, the catheter can be any suitable size, e.g., a catheter can have an outer diameter of 33 French (11 mm). On the other hand, if the delivery device could be introduced directly into the femoral vein without the need for an introducer, the delivery device itself could be as large as 30 French (10 mm).

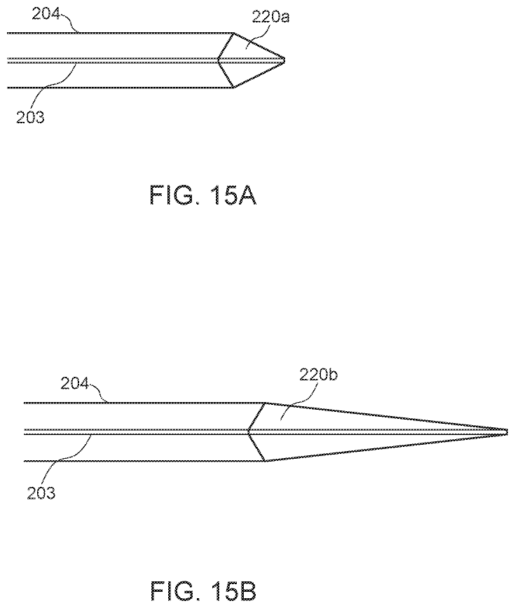

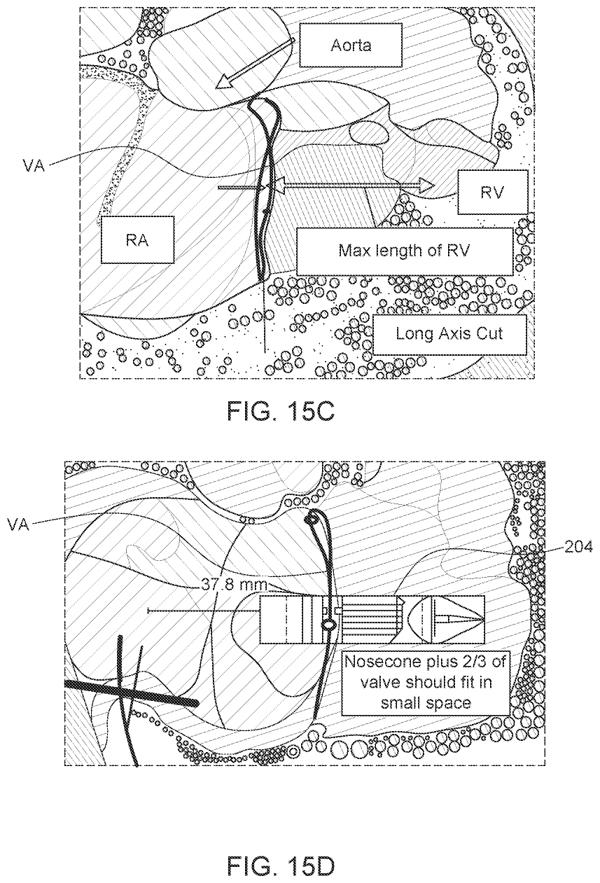

From the above description, it may seem clear that it would be beneficial to forego the use of an introducer if the goal is to maximize the size of the delivery device while still remaining within a desirable upper limit for the size of the delivery device. For example, a delivery device might be provided with a long, tapered tip or nosecone to help act as a dilator. But providing a delivery device with a long, tapered tip may cause a number of other problems. For example, FIG. 15A illustrates a simplified view of the distal end of delivery device 200, showing the valve cover 204, as well as the nosecone catheter 203 therein (which may be configured to receive a guidewire therethrough and to connect to the nosecone) and a relatively short nosecone 220a coupled thereto. The nosecone 220a may be formed from a soft, atraumatic material, while preferably allowing the nosecone 220a to be seen under imaging (e.g., fluoroscopy or echocardiography). In some embodiments, the nosecone 220a may be formed of silicone, thermoplastic polyurethane, soft Pebax, or similar materials. Radiopacity may be achieved by mixing a radiopaque agent, such as barium sulfate, into the nosecone material. Echogenicity may be achieved by adding a foaming agent to the nosecone material.

One reason that a short nosecone 220a (e.g. about 15 mm to about 20 mm in length) may be desirable is that, when the prosthetic heart valve is received within the valve cover 204 in the collapsed condition, the valve cover 204 may become quite stiff with little bendability. During delivery, the valve cover 204 and nosecone 220a will need to be steered to the annulus of the heart valve being replaced (e.g. the mitral or tricuspid annulus). Space in the heart is limited, and particularly if the valve cover 204 is relatively rigid when the prosthetic heart valve is housed therein, minimizing the length of the nosecone 220a may make steering significantly easier. However, as noted above, a short nosecone 220a may not be desirable if the goal is to eliminate the introducer/dilator. If the introducer/dilator is eliminated, it may be desirable to have a much longer, tapered nosecone such as nosecone 220b shown in FIG. 15B. For example, the femoral vein could be pre-dilated to increase the size of the femoral vein, and the nosecone 220b may be directly inserted into the femoral vein without a separate introducer sheath. The long tapered nosecone 220b may make it significantly easier to push the delivery device through the femoral vein, compared to using nosecone 220a without an introducer. However, while this long nosecone 220b may make introduction without a separate introducer easier, it may make steering within the heart significantly more difficult. Said otherwise, if it is desirable to eliminate the need for a separate introducer sheath, the length of the nosecone of the delivery device may create a problem because, while it is desirable to maximize the nosecone length for introduction and advancement through the femoral vein, it is also desirable to minimize the nosecone length for steering within the heart. It should be understood that, while the description is generally used in the context of the femoral vein, the concepts described herein may apply to other arteries or veins that need to be accessed or minimally invasive prosthetic heart valve delivery.

In addition to a long nosecone making it more difficult to appropriately steer the prosthetic heart valve into the annulus, there is another potential drawback of having a long nosecone. In particular, the amount of space available within the ventricle may be very limited. This may be particularly true for the right ventricle compared to the left ventricle (although the limited space may exist in either ventricle), and this may be particularly true for the type of patient that may require a heart valve replacement. A CT scan slice (long axis cut) of the heart is shown in FIG. 15C, which illustrates the right atrium RA, right ventricle RV, and a representation of the valve annulus VA separating the two. A double-sided arrow in FIG. 15C represents the maximum length of the right ventricle RV, and thus the maximum space available for components of a delivery device to be received within the right ventricle RV. Certain prosthetic heart valves, including one which incorporates the frame shown in FIG. 1, may generally follow what is known as the one-third, two-thirds rule. This rule (which may be better thought of as guidance) means that, while the prosthetic heart valve is still collapsed within the delivery device, approximately two-thirds of the length of the prosthetic heart valve should be positioned on the ventricle side of the valve annulus VA, and one-third of the length of the prosthetic heart valve should be positioned on the atrium side of the valve annulus VA. Using this guidance, as the prosthetic heart valve is unsheathed and deployed, the prosthetic heart valve may be allowed to expand in the desired fashion in the desired location. FIG. 15D shows a similar CT scan slice as shown in FIG. 15C, but includes a representation of the valve cover 204 (with the prosthetic heart valve collapsed therein) with about two-thirds of the length of the prosthetic heart valve positioned ventricularly and about one-third of the length of the prosthetic heart valve positioned atrially. As can be seen in FIG. 15D, which includes a more standard short nosecone, there may already be very limited clearance between the distal end of the nosecone and the wall of the ventricle. In other words, any additional length of the nosecone might otherwise result in the nosecone contacting the ventricular wall, which may make the two-thirds, one-third positioning described above impossible. Thus, a large length of the nosecone may not only make steering into the annulus difficult, but it may make it impossible to achieve the desired positioning of the prosthetic heart valve relative to the annulus pre-deployment. And although FIGS. 15C-D show the right heart, the same problem may exist in the left heart.

One solution to the above-described issues is to form the nosecone as a member that may be inflated and deflated to increase and decrease the length of the nosecone, respectively. In other words, a user may forego a separate introducer sheath that the delivery device itself needs to be passed through, and pass the delivery device through the femoral vein while the nosecone is inflated to have a long, tapered length. When the delivery device needs to be steered within the heart (e.g., after exiting the inferior vena cava for a tricuspid valve replacement, or after crossing the atrial septum for a mitral valve replacement), the nosecone may be at least partially deflated to a small length (the deflation may also decrease the stiffness of the balloon nosecone) to allow for easier maneuverability. However, changing the nosecone from a solid structure to an inflatable structure may create additional problems that need to be solved. For example, an inflatable nosecone requires a lumen to allow an inflation medium to pass into the nosecone for inflation and to be removed from the nosecone for deflation. Typically, this inflation lumen runs through a central portion of the delivery device to a port at the proximal end of the delivery device, which requires additional space, and space may be limited in such a delivery device. For the particular delivery device 200 described above, the inflation lumen would likely need to pass through the center of the coupling ring or suture ring 300. Suture ring 300, described above, may not have enough interior space to accommodate an inflation lumen without modification, and modification may be difficult while still allowing the suture ring 300 to perform its primary function. As noted above, it may also be desirable for the nosecone to be able to be coupled to the delivery device only after the prosthetic heart valve is loaded into the valve cover 204. This may be problematic because it may require the inflation lumen to be formed as two or more discontinuous lumens that can be sealingly coupled to form a single, continuous lumen through which inflation media may flow without any leaks.

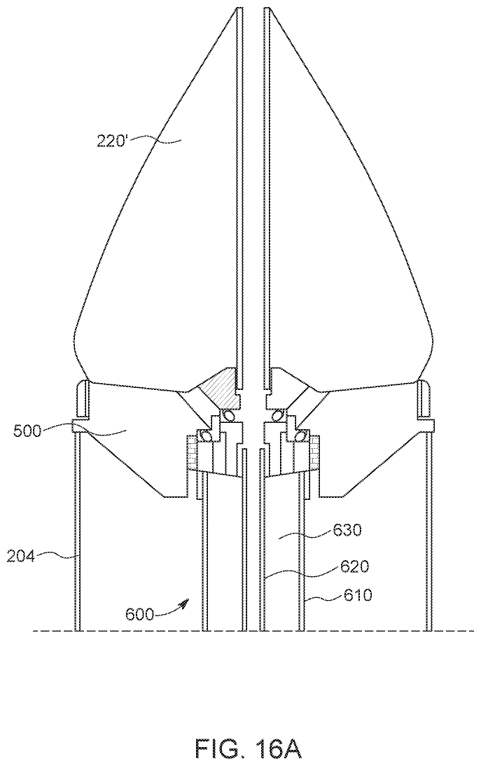

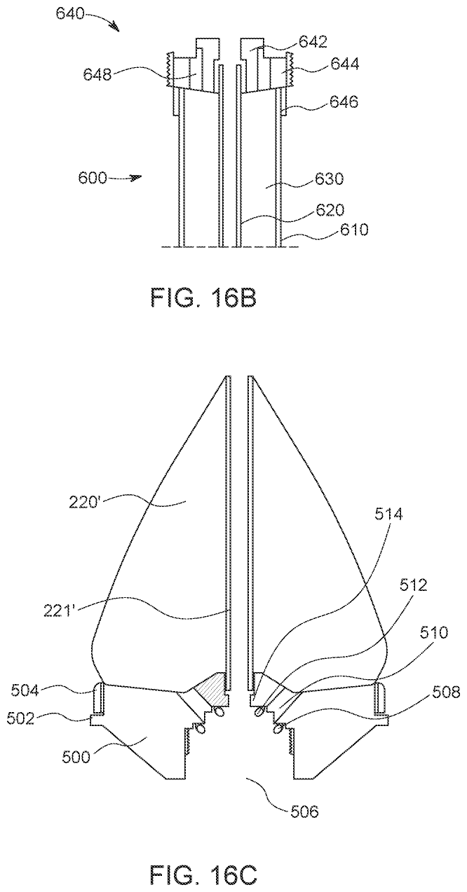

In order to allow the nosecone to be coupled to the delivery device after the prosthetic heart valve is received within the valve cover, a specialized adapter may be provided. FIG. 16A illustrates a longitudinal cross-section of a distal end of the delivery device 200 that incorporates a balloon nosecone 220′ coupled to the delivery device 200 via a specialized adapter system. The balloon nosecone 220′ preferably includes one or more lubricious coatings on an outer surface thereof to help reduce friction between the balloon nosecone 220′ and the vasculature (e.g. the femoral vein) through which it travels. For example, hydrophilic or hydrophobic coatings may be suitable, as well as silicone coatings, or other types of lubricious coatings. The balloon nosecone 220′ may be formed to have a tapered shape in which the proximal or trailing end has a relatively large diameter which tapers to a smaller diameter in the distal direction toward the leading end. The length of the balloon nosecone 220′ is partially dependent on the amount of inflation medium that is pushed into (or pulled out of) the balloon nosecone 220′, and partially dependent on the material that forms the balloon nosecone 220′ (e.g. non-compliant balloon materials may only marginally grow when inflated with higher pressure, while semi-compliant materials may grow more when inflated with higher pressure). In some examples, as the delivery device 200 is passed through the vasculature (e.g., the femoral vein) the balloon nosecone 220′ is inflated so that the length of the balloon nosecone 220′ is between about 1.5 cm and about 10 cm, although other specific lengths may be suitable depending on the particular use case. After the leading end of the delivery device 200 is ready for maneuvering to prepare for deployment of the prosthetic heart valve (e.g., when, or after, exiting the inferior vena cava for a tricuspid valve replacement, or when, or after, crossing the atrial septum for a mitral valve replacement), a volume of inflation medium may be withdrawn from the balloon nosecone 220′ to significantly decrease the length of the balloon nosecone 220′ to allow for increased maneuverability.

Still referring to FIG. 16A, the nosecone catheter 600 is shown as already being coupled to the balloon nosecone 220′ via a specialized adapter system, described in greater detail below. Only the distal end of the nosecone catheter 600 is shown in FIG. 16A, and the nosecone catheter 600 may include a portion that serves as an outer tube 610 and a portion that serves as an inner tube 620. The inner tube 610 and outer tube 620 may be formed from any suitable material, and preferably have very thin walls. For example, the tubes 610, 620, may be formed from very thing extruded materials, or dip-coated materials such as polyimide may be used. Functionally, the inner tube 620 acts as a guidewire lumen that allows a guidewire (e.g. a 0.035 inch guidewire) to pass through the nosecone catheter 600 and through a guidewire lumen of the balloon nosecone 220′ which extends through the entire length of the balloon nosecone 220′. The size of the inner tube 620 may depend on the specific function, but if intended to be used with a 0.035 inch guidewire, the inner diameter of the inner tube 620 may be slightly larger than 0.035 inches. It should be understood that other diameters may be suitable for use with differently-sized guidewires. Functionally, the outer tube 610 may serve to support a balloon nosecone adapter at a distal end thereof, as well as to create an inflation lumen 630 that is bounded on the outside by the outer tube 610 and on the inside by the inner tube 620. The inflation lumen 630 may terminate, at its proximal end (not shown) in an inflation port, for example on a handle of the delivery device 200, so that inflation medium may be pushed into, or withdrawn from, the inflation lumen 630 at the inflation port.

Referring now to FIG. 16B, which shows the distal end of the nosecone catheter 600 isolated from other components of the delivery catheter 200, and disconnected from the balloon nosecone 220′. The distal ends of the inner tube 620 and outer tube 610 may be coupled to a catheter adapter 640 which, as described in greater detail below, may be used to sealingly couple to a nosecone adapter 500 of the nosecone balloon 220′. The catheter adapter 640 may include an inner portion 642 that includes an interior shoulder that the distal end of the inner tube 620 may abut. The distal end of the inner tube 620 may be rigidly fixed, for example via adhesives, to an interior surface of the inner portion 642 of the catheter adapter 640. The inner portion 642 of the catheter adapter 640 may include a lumen or channel that is aligned with the lumen of the inner tube 630 so that the guidewire may pass through the inner tube 620, through the catheter adapter 640, and through the balloon nosecone 220′. The catheter adapter 640 may also include an outer portion 644 radially outward of the inner portion 642. The outer portion 644 may include a proximally extending flange 646. The distal end of the outer tube 610 may be in contact with the proximal end of the outer portion 644, and an outer surface of the outer tube 610 may be in contact with an inner surface of the flange 646. The outer tube 610 may be fixedly coupled to the outer portion 644 of the catheter adapter 640, for example via adhesives between the outer surface of the outer tube 610 and the inner surface of flange 646. The outer surface of the outer portion 644 may include a coupling mechanism, such as threads, that function to couple the nosecone adapter 500 (described in greater detail below) to the catheter adapter 640. One or more channels 648 may be formed between the inner portion 642 and outer portion 644 of the catheter adapter 640, the channels 648 being in fluid communication with the inflation lumen 630. Although two channels 648 are shown in FIG. 16B, a single channel 648 could be included, or more than two channels 648 could be included. If multiple channels 648 are included, they are preferably spaced evenly around the circumference of the catheter adapter 640 so that inflation medium flows relatively uniformly into (or out of) the balloon nosecone 220′.

Referring now to FIG. 16C, the balloon nosecone 220′ with nosecone adapter 500 are shown isolated from other components of the delivery catheter 200, and decoupled from the nosecone catheter 640. The nosecone adapter 500 may have an outer diameter that is about equal to the outer diameter of the valve cover 204. The nosecone adapter 500 may include a radial flange 502 that includes a proximal surface that abuts the distal end of the valve cover 204 in the delivery condition of the delivery catheter 200. The radial flange 502 may include a distal surface that may form a shoulder with the main body of the nosecone adapter 500, which may provide a convenient surface for a ring 504 to be coupled to the nosecone adapter 500. In other words, the transition between the outer surfaces of the ring 504, flange 502, and valve cover 204 may be relatively smooth without presenting any sharp or traumatic surfaces with the configuration described above. The ring 504 may be formed of a radiopaque material, such as a metal or metal alloy like tantalum or platinum-iridium, with the ring 504 functioning as a radiopaque marker to help visualize the position of the distal end of the delivery catheter 200 under fluoroscopy (or another suitable imaging modality) during delivery of the prosthetic heart valve. The ring 504 may also help avoid “peel” forces when the balloon nosecone 220′ is inflated. If the nosecone adapter 500 is formed from a plastic such as Nylon, and the balloon nosecone 220′ is formed from Nylon or Pebax, the balloon nosecone 220′ may be thermally bonded to the nosecone adapter 500, for example using a laser bond.

Still referring to FIG. 16C, the nosecone adapter 500 may define an interior channel 506 that has one or more steps in diameter. The interior channel 506 may include a large diameter portion at a proximal end, the large diameter portion having an inner diameter about equal to the outer diameter of the outer portion 644 of the catheter adapter 640. The inner surface of the nosecone adapter 500 that defines the large diameter section of the channel 506 may include a coupling mechanism, such as threads, adapted to engage the complementary coupling feature of the outer portion 644 of the catheter adapter 640. Although the coupling features are described as threads, other mechanisms such as snaps may instead be suitable. Immediately distal to the large diameter portion of the channel 506, the channel 506 may include a step-down in diameter to define a shoulder which receives a gasket 508, such as an O-ring or another seal. The channel 506 may include another step-down in diameter distal to the gasket 508, and one or more lumens 510 may be formed through the nosecone adapter 500 at that step-down in diameter, the one or more lumens 510 opening to the interior volume of the balloon nosecone 220′. The number and position of the lumens 510 should match the number and position of the channels 648 of the catheter adapter 540 so that inflation medium can readily flow between the channels 648 and the corresponding lumens 510 into or out of the balloon nosecone 220′. Distal to the step-down at the lumens 510, another step-down in diameter may be formed to create a shoulder, with another gasket 512, such as an O-ring or another seal, received at the shoulder. Finally, distal to the shoulder that receives the distal gasket 512, a flange 514 extends radially inwardly. The flange 514 may define an aperture that connects the guidewire lumen within the inner tube 620 to the guidewire lumen within the interior balloon tube 221′. The proximal end of the interior balloon tube 221′ may abut the distal face of the flange 514, and the proximal end of the interior balloon tube 221′ may be fixedly coupled to the nosecone adapter 500, for example via adhesives between the outer surface of the interior balloon tube 221′ and the inner surface of the nosecone adapter 500 distal to the flange 514. It should also be understood that the balloon nosecone 220′ itself is fixedly connected to the nosecone adapter 500, for example via adhesives. With this configuration, a sealed interior volume is created between the balloon nosecone 220′ and the distal face of the nosecone adapter 500, other than the lumens 510 which allow for inflation media to flow into or out of the otherwise sealed interior volume of the balloon nosecone 220′.

Referring back to FIG. 16A, it should be understood that the prosthetic heart valve may be loaded into the valve cover 204 while the balloon nosecone 220′ (including the nosecone adapter 500) are disconnected from the nosecone catheter 600. After the prosthetic heart valve is loaded into the valve cover 204, the nosecone adapter 500 may be coupled to the nosecone catheter adapter 640, for example via threading (as described above), via a snap-fit connection, or any other suitable connection. When coupled, as shown in FIG. 16A, the one or more channels 648 align with the one or more lumens 512, which fluidly connects the inflation lumen 630 with the balloon nosecone 220′. Upon coupling, the inner portion 642 of the catheter adapter 640 presses against gasket 512, while the outer portion 644 of the catheter adapter 640 presses against gasket 208, ensuring that the fluid pathway between the inflation lumen 630 and the balloon nosecone 220′ is sealed. In other words, the specialized adapter connection between the nosecone catheter 600 and the balloon nosecone 220′ allows for an inflation lumen to be maintained even though the balloon nosecone 220′ is not coupled to the nosecone catheter 600 until after the prosthetic heart valve is loaded into valve cover 204.

It should be understood that if delivery device 200 includes a solid nosecone that does not require an inflation lumen, the nosecone catheter may essentially be just the inner tube 620 of nosecone catheter 600, with a distal coupling attachment like a threaded insert that mates to the solid nosecone, allowing the solid nosecone to be attached to the nosecone catheter after the prosthetic heart valve is loaded into the valve cover 204. However, due to the need for an inflation lumen 630 for balloon nosecone 220′, the overall diameter of balloon nosecone 600 is larger than what would be required for a solid nosecone. If a suture rigging assembly similar to suture rigging assembly 300 is used with delivery device 200, it would be easier to fit the smaller nosecone catheter (e.g. only inner tube 620) associated with the solid nosecone through the lumen 308 of coupling ring 301 than it would be to fit the larger nosecone catheter (e.g. both inner tube 620 and outer tube 610) required for the balloon nosecone 220′ through that same lumen 308 of coupling ring 301. If the inner diameter of the lumen 308 were increased to accommodate the larger nosecone catheter 600, there may not be enough space to include the desired number and configuration of bores 316 (e.g., inner ring 318 and outer ring 320) without increasing the outer diameter of the head or hemispherical surface 314. For example, referring to FIG. 5, any increase in the diameter of the lumen 308 would leave less space for bores 316. Increasing the diameter of the hemispherical surface 314 to allow for the desired number and configuration of bores 316 may also not be desirable, as increasing the size of the coupling ring 301 may require other components to increase in size, and thus lead to an overall size of the delivery device 200 that is larger than desired. Thus, in order to have the functionality of coupling ring 301 while also having the functionality of the balloon nosecone 220′, alternate designs for coupling ring 301 may be required.

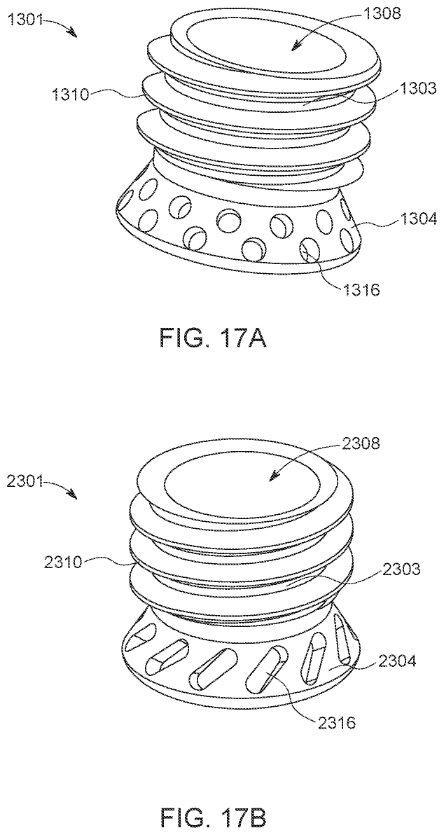

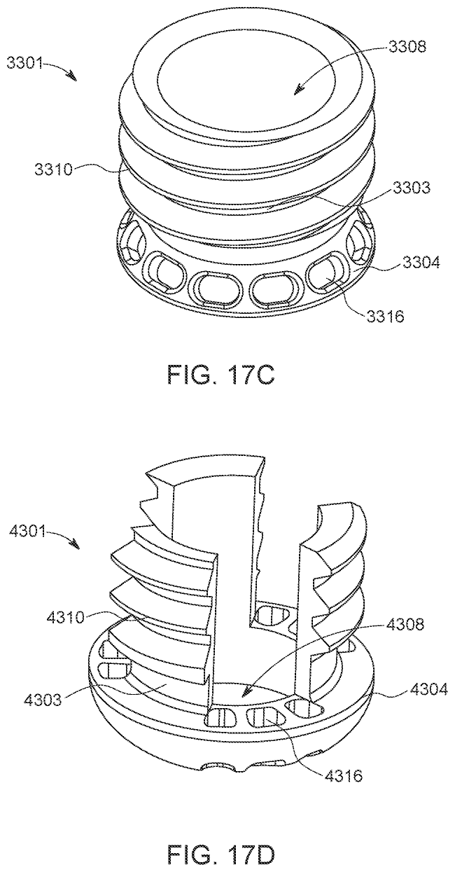

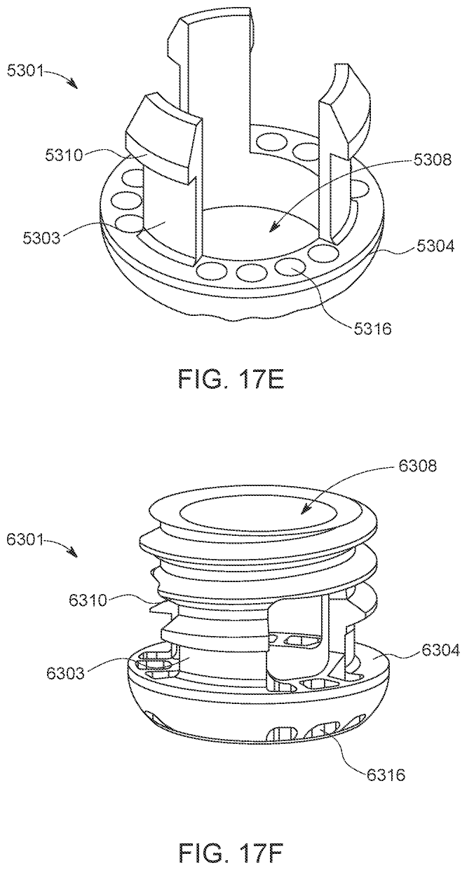

FIGS. 17A-F are perspective views of suture rings 1301, 2301, 3301, 4301, 5301, and 6301 that are alternate versions of suture ring 301. The common feature among the alternate suture rings of FIGS. 17A-F is that each of the suture rings includes an interior lumen 1308, 2308, 3308, 4308, 5308, and 6308 that is larger in inner diameter than lumen 308 of suture ring 301, while having a maximum outer diameter that is no larger than the maximum outer diameter of suture ring 301.

Referring to FIGS. 17A-C, each suture ring 1301, 2301, 3301 generally includes a cylindrical body 1303, 2303, 3303 with external threads 1310, 2310, 3310 on an outer surface thereof. These components may be substantially similar to the corresponding components of suture ring 301, with the main distinction being that interior lumens 1308, 2308, 3308 have a larger diameter than interior lumen 308. One reason that the larger interior lumen 1308, 2308, 3308 is possible, without increasing the maximum diameter of the suture ring 1301, 2301, 3301 compared to suture ring 301, is that instead of a head 304 with a mushroom shape or hemispherical surface 314, the distal ends of the suture rings 1301, 2301, 3301 have an outwardly tapered surface. For example, each suture ring 1301, 2301, 3301 has a head 1304, 2304, 3304 that starts at the distal end of the cylindrical body 1303, 2303, 3303 and increases in diameter (in a generally frustoconical shape) toward the distal-most end of the suture ring 1301, 2301, 3301. With this configuration each of the bores 1316, 2316, 3316 extends from the interior to exterior surface of the obliquely angled tapered head 1304, 2034, 3304, compared to bores 316 that extend only in the proximal-to-distal direction through head 304. Because of this altered positioning of the bores 316, the desired number of bores 1316, 2316, 3316 may be created in the head 1304, 2304, 3304 without needing to enlarge the maximum diameter of the suture ring 1301, 2301, 3301. The precise shape and configuration of the bores 1316, 2316, 3316 may be varied. For example, suture ring 1301 includes a proximal and distal row of staggered bores 1316 having a generally circular shape, with each row having twelve bores 1316 similar to suture ring 301. With this configuration, each bore 1316 may receive one suture thread 400. On the other hand, suture rings 2301 and 3301 may each include a single row of relatively large bores 2316, 3316, for example twelve in total, with each bore 2316, 3316 adapted to receive two sutured threads 400. Bores 2316 may be generally oblong or elliptical, with the long axes of the bores 2316 generally obliquely angled relative to the proximal-to-distal direction along the head 2304. Bores 3316 may also be generally oblong or elliptical, but with the long axes of the bores 3316 generally oriented perpendicularly to the proximal-to-distal direction along the head 3304.

While suture rings 1301, 2301, 3301 are able to create more space for the bores 1316, 2316, 3316 by having a generally frustoconical head 1304, 2304, 3304, suture rings 4301, 5301, 6301 of FIGS. 17D-F maintain a generally mushroom or hemispherically-shaped head 4304, 5304, 6304 but are able to create additional space for the bores 4316, 5316, 6316 by creating strategically placed windows. In other words, suture rings 4301, 5301, 6301 include larger interior diameter lumens 4308, 5308, 6308 without needing to increase the maximum diameter of the suture rings 4301, 5301, 6301 compared to suture ring 301. For example, suture rings 4301, 5301 do not include a continuous cylindrical body, but rather include a plurality of individual upstanding posts 4303, 5303 that lie within a cylindrical surface, but have discontinuities or windows between circumferentially adjacent posts 4303, 5303. The various bores 4316, 5316 may be created in the proximal-to-distal direction of the heads 4304, 5304 in the open areas between circumferentially adjacent posts 4303, 5303. The posts 4303 of suture ring 4301 may include external threads 4310 to connect to the delivery device 200, whereas the posts 5303 of suture ring 5303 may include protrusions 5310 that may have a snap-fit to connect to the delivery device 200.

Suture ring 6301 may be thought of as a hybrid of suture ring 4301 and suture ring 301. In other words, suture ring 6301 includes the larger lumen and a proximal portion that is a fully cylindrical body, with the fully cylindrical body leading to posts 6303 similar to posts 4303. The bores 6316 may extend in the proximal-to-distal direction through the head 6304 adjacent the openings, windows, or cutouts between circumferentially adjacent posts 6303 and distal to the fully cylindrical body portion. In the illustrated embodiment, suture ring 6301 includes external threads 6310 to couple to the delivery device.

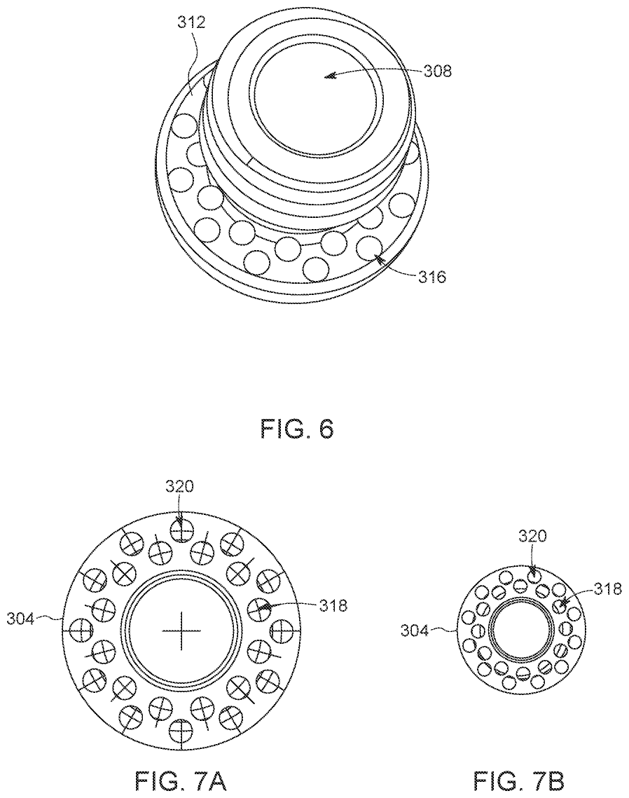

As explained above, the suture rings shown and described in connection with FIGS. 17A-F allow for a larger interior lumen without increasing the maximum diameter of the suture ring, which in turn allows for the larger nosecone catheter 600 to be used for a balloon nosecone 220′ without needing to increase the size of any components of the delivery system. The use of the balloon nosecone 220′, in turn, may allow for the delivery device 200 to be used without a separate introducer, which may allow for an overall smaller profile of delivery components entering the body. However, the suture rings shown and described in connection with FIGS. 17A-F may provide additional benefits. For example, suture ring 301 includes a mushroom or hemispherical-shaped head 304 with a full cylindrical body 303 leading to the head 304. The bores of the suture rings of FIGS. 17A-F are easily accessible from each side of the hole. For suture rings 1301, 2301, 3301, the frustoconical heads provide easy access to each side of the bores. For suture rings 4301, 5301, 6301, the open space or windows adjacent to the bores similarly allow for easy access to each side of the bores. This easy access makes manufacturing the bores with chamfered surfaces significantly easier compared to creating chamfers in the bores 316 of suture rings 301. For example, referring to FIG. 6, the particularly the inner row of bores 316 are very close to the cylindrical body 303, and thus there may simply not be enough working space to create chamfered surfaces (or rounded edges) in the bores 316. Suture rigging assembly 300 solves this problem, at least in part, by the use of a padding suture thread 402, which is an additional suture thread that directly sits on the shoulder 312, so that suture threads 400 extending through the bores 316 loop over the padding suture thread 402. This padding suture thread 402 helps avoid direct contact between the suture threads 400 and any sharp surfaces that may be present at the bores 316. However, because the suture rings shown and described in connection with FIGS. 17A-F include bores that can be easily accessed from both sides, rounded surfaces or chamfers may be created leading into the bores so that there are no sharp surfaces that may risk damaging suture thread 400, without the need for an additional padding suture thread 402.

Although specific embodiments of suture rings are shown and described in connection with FIGS. 17A-F, it should be understood that various features may be mixed and matched between the embodiments, including choosing threads for connection to the delivery device, snaps for the connection to the delivery device, or other connection mechanisms. Further, it should be understood that the description of the function of suture ring 301 applies with equal force to the suture rings shown and described in connection with FIGS. 17A-F, other than the differences explicitly noted herein.

Still further, although the inflatable balloon nosecone described above is described in the context of a delivery device or system for delivering prosthetic mitral or tricuspid valves, it should be understood that the inflatable balloon nosecone may be used in delivery systems for delivering other devices into a patient in a minimally invasive manner. For example, a delivery device that incorporates the inflatable balloon nosecone (including the ability to inflate or deflate the nosecone at different stages of delivery) may be used with other prosthetic heart valves, including aortic and pulmonary valves, with other devices for implantation into the heart, such as PFO or LAA occluders and/or pacemakers (or portions thereof), with other implantable devices such as vena cava filters, stents, etc. It should further be understood that a delivery system that incorporates the inflatable nosecone described herein may be used with delivery devices that do not include implantable devices, such as embolic protection filter devices or hydrodynamic thrombectomy systems.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A delivery system for delivering a medical device, the delivery system comprising:
a delivery device including:
an outer delivery sheath having a compartment at a distal end thereof, the compartment configured to receive the medical device therein;
a nosecone catheter positioned radially within the outer delivery sheath, the nosecone catheter having an inner tube configured to receive a guidewire therethrough, and an outer tube, an inflation lumen defined between the inner tube and the outer tube; and
a nosecone that has a length that may be adjusted by pushing an inflation medium into the balloon nosecone or withdrawing the inflation medium from the balloon nosecone via the inflation lumen, the balloon nosecone being reversibly coupled to the nosecone catheter.

2. The delivery system of claim 1, wherein the medical device is a prosthetic heart valve, and the compartment is a valve cover configured to receive the prosthetic heart valve while the prosthetic heart valve is in a collapsed condition.

3. The delivery system of claim 2, wherein the prosthetic heart valve is configured to be loaded into the valve cover in the collapsed condition while the nosecone is decoupled from the nosecone catheter, and the nosecone is configured to be coupled to the nosecone catheter after the prosthetic heart valve is loaded into the valve cover.

4. The delivery system of claim 2, wherein the nosecone is formed as a balloon, the balloon having a proximal end fixed to a nosecone adapter.

5. The delivery system of claim 4, wherein the nosecone catheter includes a catheter adapter at a distal end thereof, the catheter adapter configured to releasably couple to the nosecone adapter.

6. The delivery system of claim 5, wherein the catheter adapter and the nosecone adapter are configured to releasably couple via a threaded mechanism.

7. The delivery system of claim 5, wherein the catheter adapter includes a channel extending therethrough, the channel being in fluid communication with the inflation lumen.

8. The delivery system of claim 7, wherein the nosecone adapter includes a lumen extending therethrough and leading into an interior volume of the nosecone, the channel being in fluid communication with the lumen when the catheter adapter is coupled to the nosecone adapter.

9. The delivery system of claim 8, wherein when the catheter adapter is coupled to the nosecone adapter, a first gasket is positioned between the nosecone adapter and the catheter adapter, and a second gasket is positioned between the nosecone adapter and the catheter adapter, the first and second gaskets positioned on opposite sides of the channel and on opposite sides of the lumen.

10. The delivery system of claim 2, wherein the delivery device further includes a suture catheter positioned radially within the outer delivery sheath and at least partially surrounding the nosecone catheter, the suture catheter having a tip ring at a distal end thereof.

11. The delivery system of claim 10, further comprising a suture ring, the suture ring defining an interior lumen and a plurality of bores, one or more suture threads extending through the plurality of bores.

12. The delivery system of claim 11, wherein when the prosthetic heart valve is received within the valve cover in the collapsed condition, the one or more suture threads are reversibly coupled to the prosthetic heart valve.

13. The delivery system of claim 11, wherein the suture ring is configured to reversibly couple to the tip ring.

14. The delivery system of claim 13, wherein when the suture ring is coupled to the tip ring, both the inner tube and the outer tube of the nosecone catheter pass through the interior lumen of the suture ring.

15. The delivery device of claim 11, wherein the suture ring includes a cylindrical body portion and a distal head, the distal head having a generally frustoconical shape that increases in diameter in a distal direction, the plurality of bores being formed in the distal head.

16. The delivery device of claim 11, wherein the suture ring includes a cylindrical body portion and a distal head, the distal head having a generally hemispherical shape, recesses being formed at locations in the cylindrical body at spaced distances in a circumferential direction of the cylindrical body portion, each of the plurality of bores being formed in the distal head adjacent a corresponding one of the recesses.

17. A method, comprising:
introducing a delivery catheter into a femoral vein of a patient while a prosthetic heart valve is contained in a collapsed condition within a valve cover of the delivery catheter;
advancing the delivery catheter through the femoral vein toward a right atrium of the patient while a nosecone at a leading end of the delivery catheter has a first length;
continuing to advance the delivery catheter through the femoral vein until the nosecone approaches the right atrium;
after the delivery catheter approaches the right atrium, withdrawing a volume of inflation medium from the nosecone so that the nosecone has a second length smaller than the first length;
while the nosecone has the second length, maneuvering the valve cover to a position within or adjacent to an atrioventricular valve of the patient; and
after maneuvering the valve cover to the position within or adjacent to the atrioventricular valve of the patient, deploying the prosthetic heart valve into the atrioventricular valve.

18. The method of claim 17, wherein the prosthetic heart valve is a prosthetic mitral valve, and the method further comprises:
advancing the delivery catheter through the right atrium and across an atrial septum while the nosecone has the first length; and
wherein withdrawing the volume of inflation medium from the nosecone is not performed until the delivery catheter crosses the atrial septum.

19. The method of claim 17, wherein the prosthetic heart valve is a prosthetic tricuspid valve, and the volume of inflation medium is withdrawn from the nosecone upon or immediately after the delivery catheter enters the right atrium.

20. The method of claim 17, further comprising:
prior to introducing the delivery catheter into the femoral vein, loading the prosthetic heart valve into the valve cover while the nosecone is decoupled from the delivery catheter; and
after the prosthetic heart valve is loaded into the valve cover, coupling the nosecone to the delivery catheter.

21. The method of claim 17, wherein introducing the delivery catheter into the femoral vein is performed without passing the delivery catheter through an introducer catheter that has previously been introduced into the femoral vein.
